(19) United States
(12) Patent Application Publication
(10) Pub. No.: US 2010/0125324 A1
(43) Pub. Date: May 20, 2010

(54) Catheter Inner Member

(75) Inventors: Chris Collins, Petaluma, CA (US); Don Tran, Novato, CA (US); Bijan Nafea, Santa Rosa, CA (US); William Berthiaume, Santa Rosa, CA (US); Jeremy Kinkade, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/271,524

(22) Filed: Nov. 14, 2008

(51) Int. Cl. A61F 2/84; A61M 25/14

(52) U.S. Cl. 623/1.11; 604/246

(57) ABSTRACT

A catheter inner member includes an inner member body, including a first side and a second side, the second side opposing the first side. The inner member body includes an inner surface defining an inner member inner lumen and an outer surface defining a surrounding environment. The inner member further includes at least a first flush port and a second flush port in the inner member body. The first flush port and second flush port provide fluid communication between the inner lumen and a surrounding environment. The first flush port disposed on the first side and the second flush port disposed on the second side, wherein the first flush port is axially displaced from the second flush port.

### TECHNICAL FIELD

The technical field of this disclosure is medical devices, particularly, a catheter inner member for a rapid exchange catheter system for delivering a stent.

### BACKGROUND

Catheters are used in a number of medical procedures to deliver medical devices to a target site within a body and other purposes. Catheters for delivering self expanding medical devices, such as stents, typically, have a tubular shaft defining an inner lumen with an inner wall of the catheter for a number of purposes, such as holding self expanding devices constrained or the like. Catheters for delivering self expanding stents also typically have a guidewire lumen fully or partially separate or apart from the inner lumen constraining the self expanding device to be delivered. Prior to insertion into the body, the catheter lumens should be flushed with a fluid, often saline, particularly in cases where the stent is to be implanted in the carotid artery which leads to the brain, where air bubbles may lead to brain injury, such as a stroke. Flushing a rapid exchange self expanding stent delivery catheter is currently a multi-step process where pressurized fluid sources are attached to the catheter handle or shaft through rudimentary or customized attachments to flush the catheter, and any devices attached to or contained in the catheter, such as stents. In an initial pre-flush configuration the guide wire lumen is plugged by a mandrel and fluid injected into the delivery system handle when it reaches the distal end of the catheter is prevented from flowing into the guidewire lumen by the mandrel and therefore flow around the guidewire lumen and bathes the stent with fluid. Once fluid is observed having crossed the stent and started leaking from the catheter sheath, the mandrel is removed and a finger is placed over the rapid exchange guidewire exit port, and further flushing is undertaken causing flushing fluid to flow into and through the guidewire lumen and out the end of the catheter Furthermore, a full flush of the catheter is difficult to achieve based on the small spaces and small diameter lumens into and through which the fluid must travel to achieve complete elimination of air from the catheter. Catheters are long, narrow structures, and ensuring that the fluid flows through and fills all appropriate structures can require an undesirably high level of force (pressure) to be applied (to an injection syringe, for example) or while maintaining a lower force (pressure) causes an undesirable delay for the fluid to reach and fill all catheter spaces during the pre-op routine.

Additionally, catheter design is plagued by the contrasting demands of sturdiness and flexibility. Often catheters are designed with a hypotube inner member to provide sturdiness and resistance to radial collapse and compressive buckling, but the ‘sturdy’ hypotube undesirably resists bending. This sturdiness translates into reduced navigability when traversing tortuous body vessels, such as vasculature. In contrast, polymeric inner members lack the structural strength of hypotubes, while featuring improved navigation capacity due to their flexibility and ability to bend.

It would be desirable to overcome the above disadvantages.

### SUMMARY OF THE INVENTION

One aspect according to the present invention provides a catheter inner member that includes an inner member body, including a first side and a second side, the second side opposing the first side. The inner member body includes an inner surface defining an inner member inner lumen and an outer surface defining a surrounding environment. The inner member further includes at least a first flush port and a second flush port in the inner member body. The first flush port and second flush port provide fluid communication between the inner lumen and a surrounding environment. The first flush port disposed on the first side and the second flush port disposed on the second side, wherein the first flush port is axially displaced from the second flush port.

Another aspect according to the invention provides an inner member body defining an inner member inner lumen and a surrounding environment. The inner member body further includes at least a first partial circumferential cut providing fluid communication between the inner member inner lumen and a surrounding environment.

Another aspect according to the invention provides a catheter system that includes a catheter including a catheter body defining a catheter inner lumen and a surrounding environment. The system further includes an inner member maintained within the catheter inner lumen. The inner member includes an inner member body having a first side and a second side, the second side opposing the first side. The inner member body defines an inner member inner lumen and an outer surface. Furthermore, the inner member body includes at least a first flush port and a second flush port, the first flush port and second flush port providing fluid communication between the catheter inner lumen and the inner member inner lumen. The first flush port is disposed on the first side and the second flush port is disposed on the second side. The first flush port is axially displaced from the second flush port.

Yet another aspect according to the invention provides a catheter system that includes a catheter defining a lumen, and a dual lumen inner member within the catheter. The dual lumen inner member defines a first lumen and a second lumen, the first lumen parallel with the second lumen. The first lumen is in fluid communication with a distal tip of the catheter, and the second lumen is in fluid communication with a proximal end of the catheter. The second lumen includes at least a first flush port and a second flush port in the inner member body, the first flush port and second flush port providing fluid communication between the inner lumen and the surrounding environment, the first flush port disposed on the first side and the second flush port disposed on the second side, and wherein the first flush port is axially offset from the second flush port.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIG. 1A is a top view of a portion of an inner member contained within the distal portion of a catheter;

FIG. 1B is a cross section view of the inner member of FIG. 1A taken at A-A;

FIG. 1C is a cross section view of the inner member of FIG. 1A taken at B-B;

FIG. 1D is a cross section view of the inner member of FIG. 1A taken at C-C;

FIG. 1E is a nominal cross section view of the inner member of FIG. 1A;

FIG. 1F is a side cross sectional view of an inner member of FIG. 1A contained within a catheter, and lines with arrow heads illustrating fluid flow in and out of the inner member;

FIG. 1G is a close-up view of a portion of FIG. 1F with lines and arrowheads illustrating fluid flow through and out of an inner member body;

FIG. 2 is a view of a first flush port;

FIG. 3 is a view of a second flush port;

FIG. 4A is a side view of another embodiment of a catheter inner member;

FIG. 4B is a schematic diagram with lines and arrowheads illustrating fluid flow in and out of the catheter inner member of FIG. 4A;

FIG. 5 schematically illustrates a cross sectional side view of a another catheter system;

FIG. 5A illustrates the proximal portion of catheter of FIG. 5;

FIG. 5B illustrates the portion of catheter of FIG. 5 immediately distal to the portion illustrated in FIG. 5A;

FIG. 5C illustrates the portion of catheter of FIG. 5 between that illustrated in FIGS. 5B and 5D;

FIG. 5D illustrates the distal portion of catheter of FIG. 5,

FIG. 6 is a cross section view of a dual lumen inner member taken at A-A of FIG. 5B;

FIG. 7 is a cross section view of a dual lumen inner member taken at B-B of FIG. 5C; and

FIG. 8 is a flowchart of a method of flushing a catheter.

### DETAILED DESCRIPTION

Embodiments according the invention will now be described by reference to the figures wherein like numbers refer to like structures. The terms “distal” and “proximal” are used herein with reference to the treating clinician during the use of the catheter system: “distal” indicates an apparatus portion distant from, or a direction away from the clinician and “proximal” indicates an apparatus portion near to, or a direction towards the clinician.

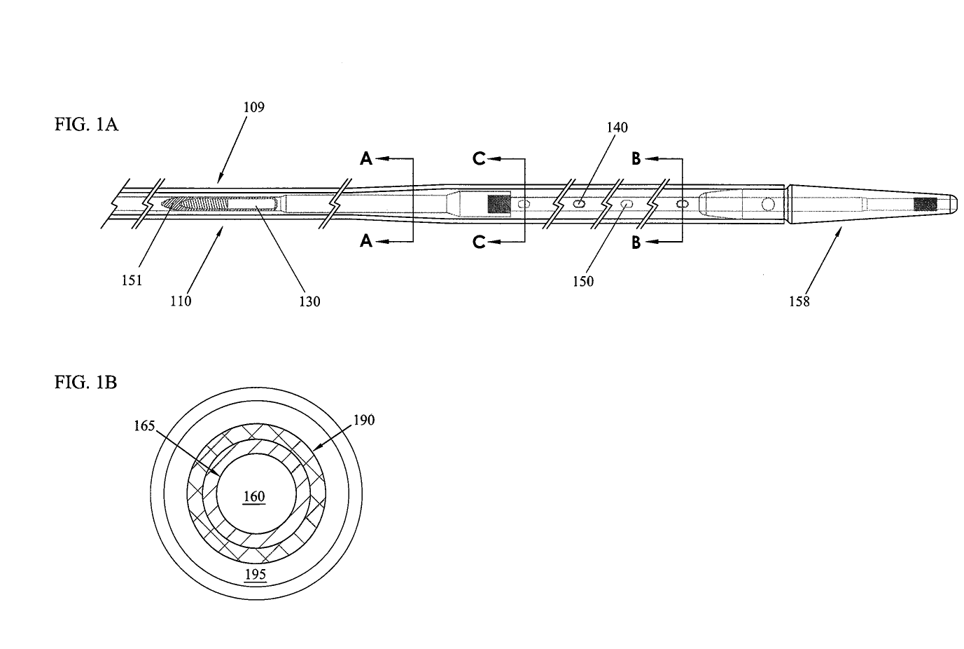

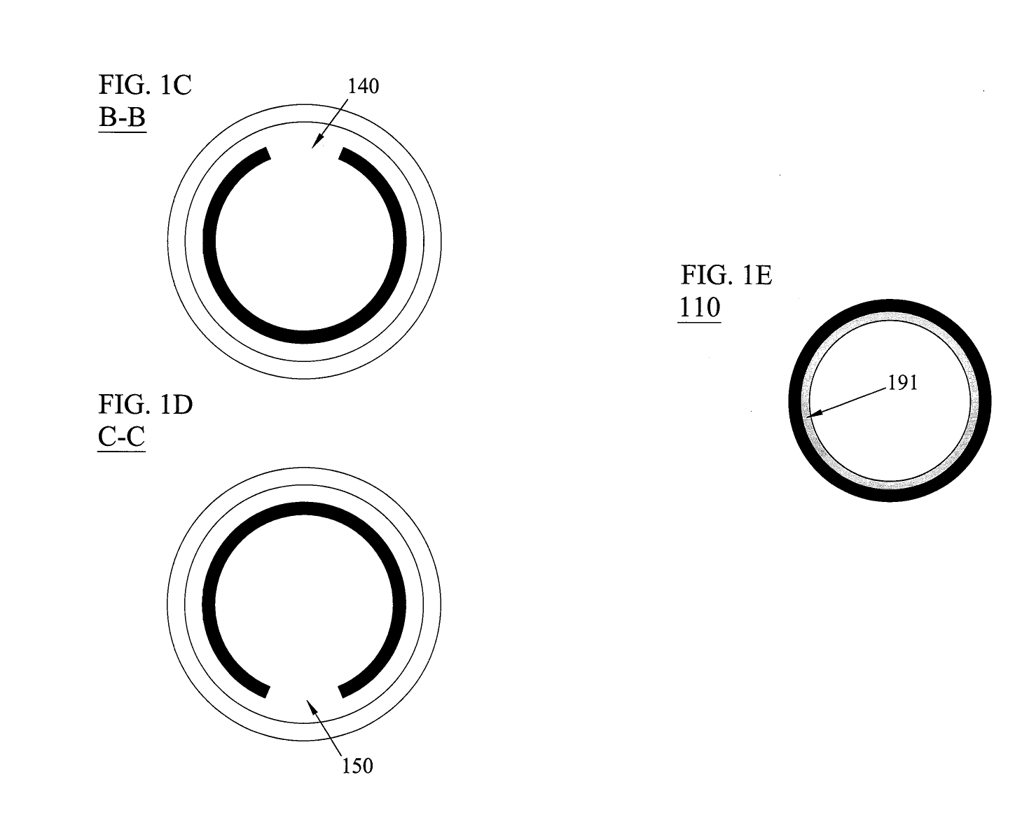

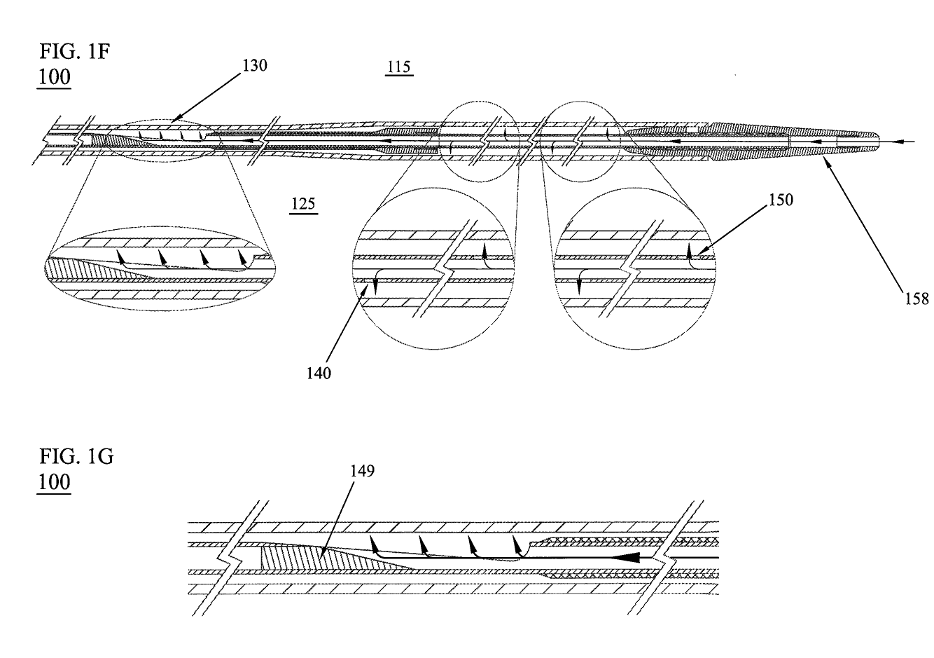

FIGS. 1A, 1B, 1F, and 1G illustrate one embodiment of an inner member 100. FIG. 1A illustrates a top view, while FIG. 1F illustrates a side view within a catheter (sheath for a self expanding stent system), FIG. 1B illustrates a cross section taken at line A-A, FIG. 1C illustrates a cross section taken at line B-B, and FIG. 1D illustrates a cross section taken at line C-C. Inner member 100 includes an inner member body 110 and a rapid exchange entrance 130. Inner member 100 includes a first side (top) 115 and a second side (bottom) 125. The second side 125 is opposite the first side 115. Inner member body 110 further includes an inner surface 165 (FIG. 1B) that defines an inner lumen 160 (FIG. 1B) and an outer surface 190 (FIG. 1B) defining the inner limits of a surrounding environment 195 (FIG. 1B). When a catheter (e.g., outer cover 109) surrounds the inner member 100, the surrounding environment is the space between the inside wall of the catheter and the outer surface 190.

Additionally, inner member body 110 includes at least a first flush port 140 and a second flush port 150. The first flush port 140 and second flush port 150 provide fluid communication between the inner lumen 160 and a surrounding environment 195. The first flush port 140 is disposed on the first side 115 and the second flush port 150 is disposed on the second side 125. In one embodiment, the first flush port 140 is axially displaced from the second flush port 150 along axis 151. The inner surface 165 can be coated with a polymer coating 191 (FIG. 1E), such as PEBAX, in certain embodiments. As an example, the inner member body 110 can be a hypotube, such as a metallic hypotube.

The inner member further includes a receptacle for receiving flushing fluid at the distal end of the inner member 110 at tip 158. The flushing fluid can be any appropriate flushing fluid, such as saline. A portion of a catheter system for delivering a self expanding stent is schematically depicted in FIG. 1F. The outer cover (catheter) 109 is disposed over a self expanding stent (not shown). To flush the system, fluid is provided to the tip 158 and flushing fluid flows through the inner member inner lumen until the fluid reaches the first opening, e.g., the first flush port 140 in the inner member and subsequent openings. A clinician can cover, or pinch, the rapid exchange entrance 130, to force fluid to flow further through the inner member and into the catheter inner lumen outside the inner member to fill all the space in the inner member inner lumen and the catheter inner lumen with flushing fluid. To deploy the stent (not shown), the physician holds the handle (not shown) and retracts the thumb button, which is attached to the outer cover to retract it relative to the inner member and thereby release the stent as is well known in the art.

FIG. 1F illustrates fluid flow through an inner member body, such as inner member body 110, in accordance with one embodiment. FIG. 1F is specifically a cutaway side view showing detail of the fluid exit from the flush ports as well as the rapid exchange port. Included in FIG. 1F are three close-up views, including a close-up view of the rapid exchange port, and two close-up views at flush ports. Each flush port is located where a stent (not shown in FIG. 1F) would be positioned in the catheter prior to deployment such that the fluid flowing through each flush port will flush the stent.

FIG. 1G illustrates fluid flow through an inner member body, such as inner member body 110, in accordance with one embodiment of the invention. Specifically, FIG. 1G illustrates a cutaway side view illustrating a rapid exchange port. FIG. 1G illustrates block 149 disposed within the inner member and providing a fluid seal to reduce flow of the flushing fluid proximal of the rapid exchange port. The block 149 can be any appropriate material, such as an adhesive, polymer, or metal configured to substantially reduce fluid flow past the block. As fluid flows from the tip of the inner member through the lumen and out the rapid exchange port, the block 149 directs any fluid that traverses the distance from the tip to the rapid exchange port out of the inner member via the rapid exchange port. FIG. 1G also illustrates the fluid flow exiting the inner member via the rapid exchange port.

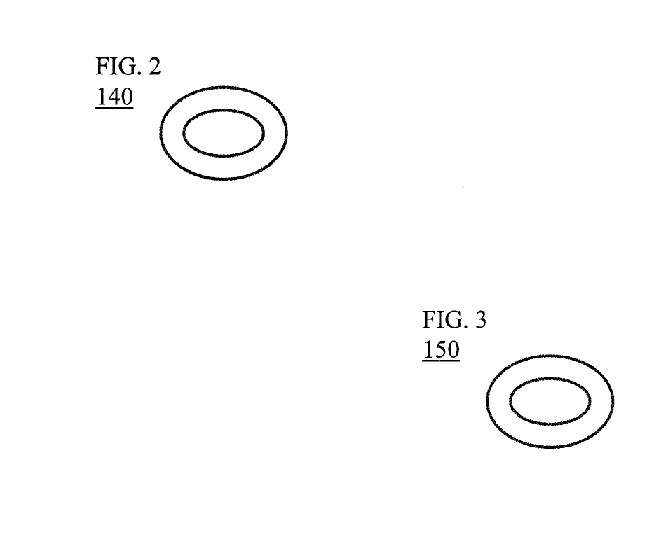

FIGS. 2 and 3 are plan views of flush ports such as 140 and 150 in FIG. 1F.

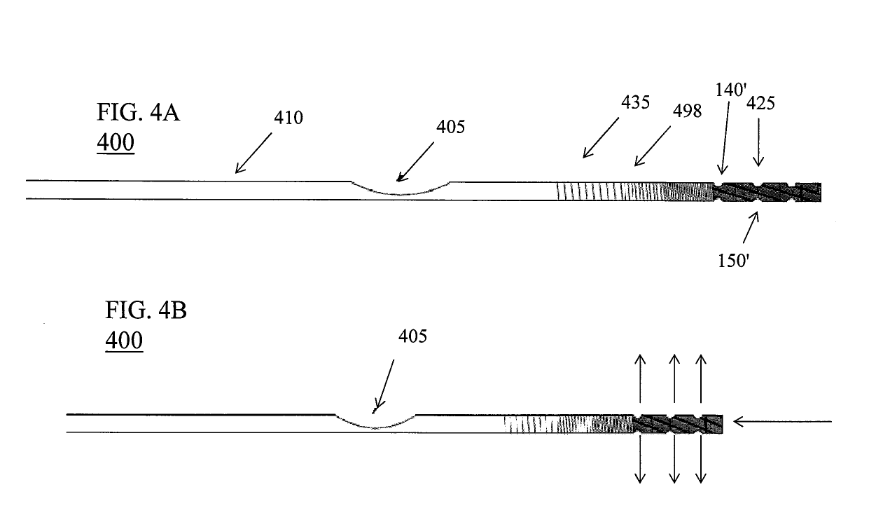

FIG. 4A is a side view of another embodiment of a catheter inner member 400 including an inner member body 410. The inner member is, in one example, a hypotube, with or without an internal polymer coating, such as PEBAX. Inner member 400 includes rapid exchange port 405. Inner member body 410 defines an inner lumen within the inner member body 410 and a surrounding environment outside the inner member body 410. When inner member body 410 is within a catheter, the surrounding environment is defined by the space between the inner wall (surface) of the catheter and the outer surface of the inner member body.

Additionally, inner member body 410 includes at least a first incomplete circumferential cut 498. An incomplete circumferential cut is a cut into the wall of the inner member body, but not around the whole circumference, thus leaving an un-cut connection between inner member body sections on each side of the cut. Any number of incomplete circumferential cuts may be included along the length of the inner member body. Such circumferential cuts may extend radially about the body, or extend spirally about the body. Such circumferential cuts can be through cuts, extending through the inner member body, providing fluid communication between the inner lumen and a surrounding environment. Alternatively, such circumferential cuts can extend from the outer surface radially a fraction of the thickness of the inner member so that the circumferential cuts do not provide fluid communication between the inner lumen and the surrounding environment, but do provide reduced resistance to bending. Furthermore, a single inner member body can have varying circumferential cuts, with some cuts radial, some cuts spiral, some cuts through cuts, and other cuts only partial.

Additionally, inner member body 410 includes a flush port portion 425 and a cut portion 435, wherein the cut portion 435 is axially offset from the flush port portion 425. Further, inner member body 410 includes a plurality of flush ports axially offset from each other and disposed on opposing sides of the inner member body 410. For example, each flush port is implemented as flush ports 140′ or 150′, similar to flush ports 140 and 150 described with reference to FIGS. 1A-1D and inner member 100.

The lumen of inner member body 410 is in fluid communication with a fluid source for providing a flushing fluid, such as a saline solution. The cut portion is disposed between the fluid source and the flush port portion.

FIG. 4B is a schematic illustration where the arrows designate the direction of fluid flow through the inner member 400, and through the flush port portion 425.

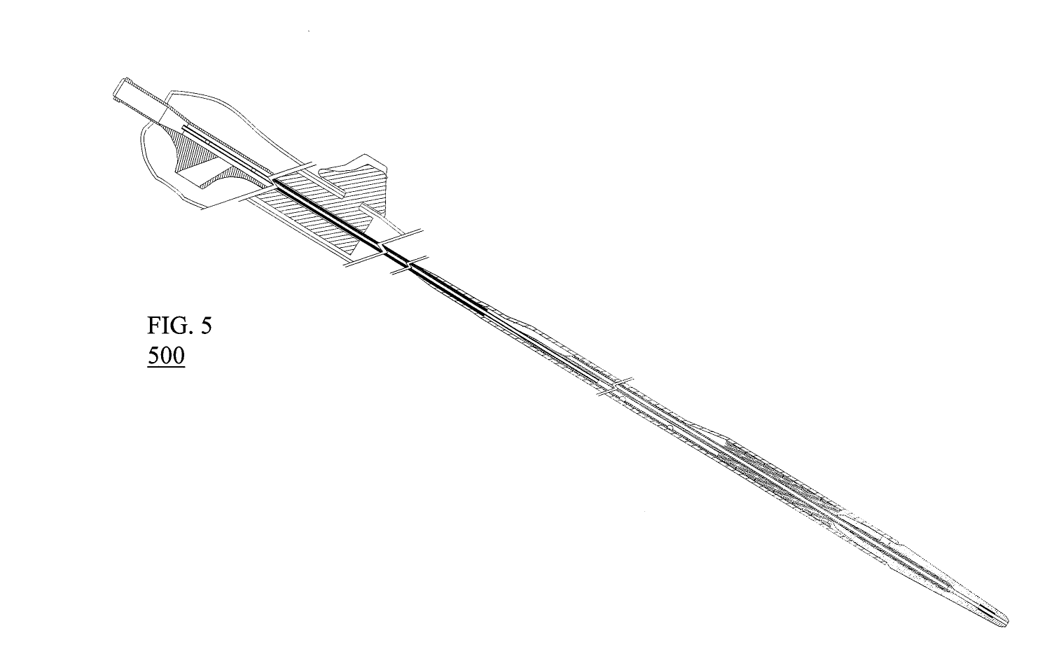

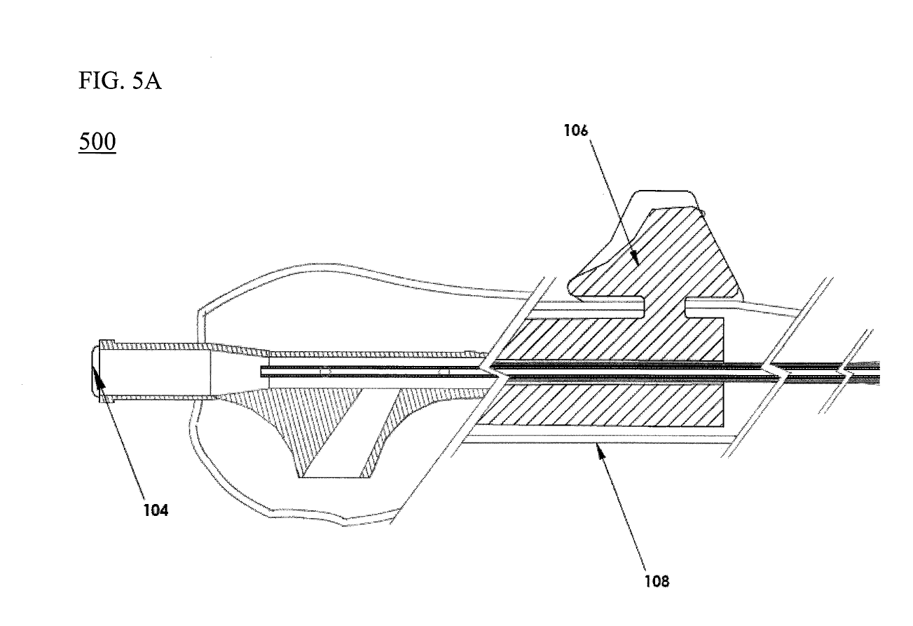

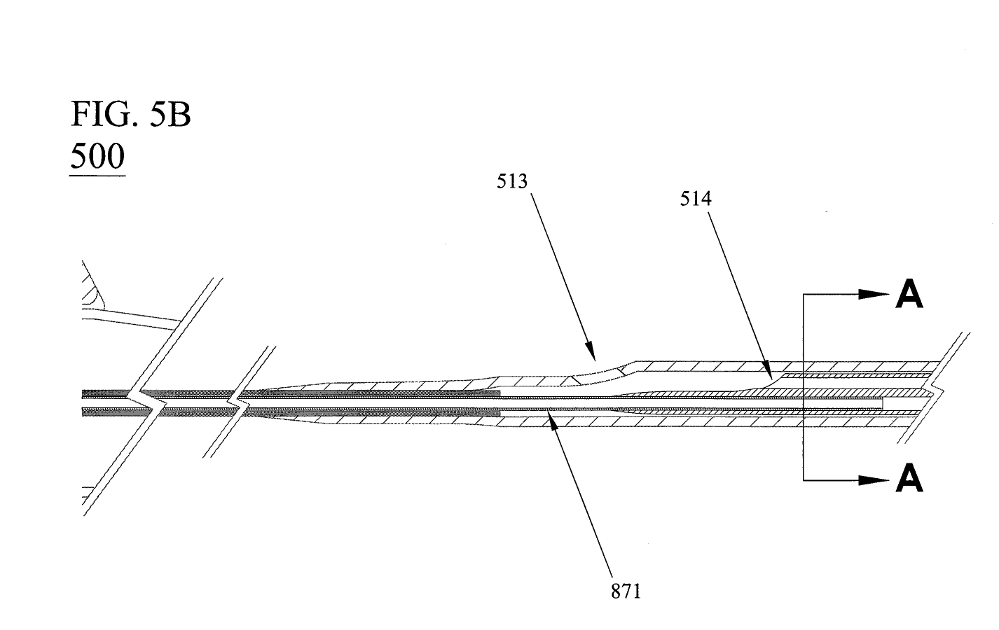

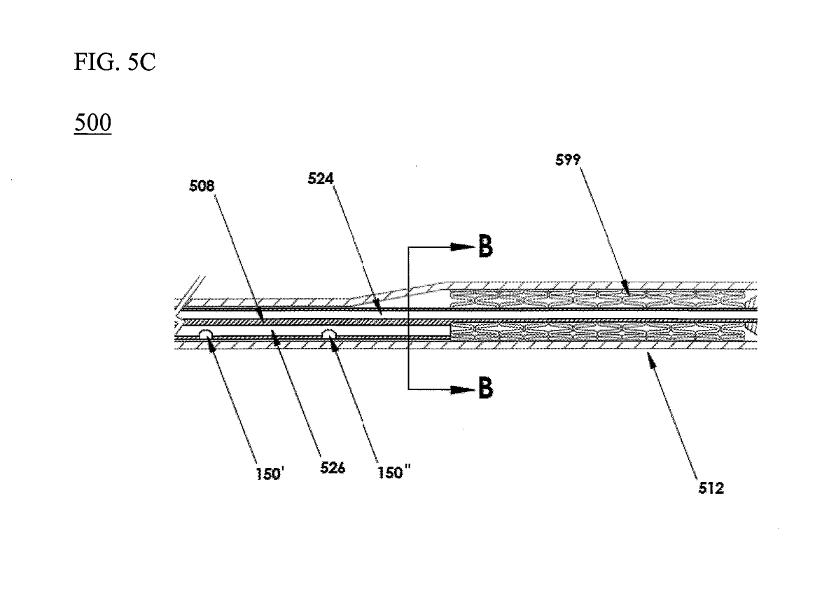

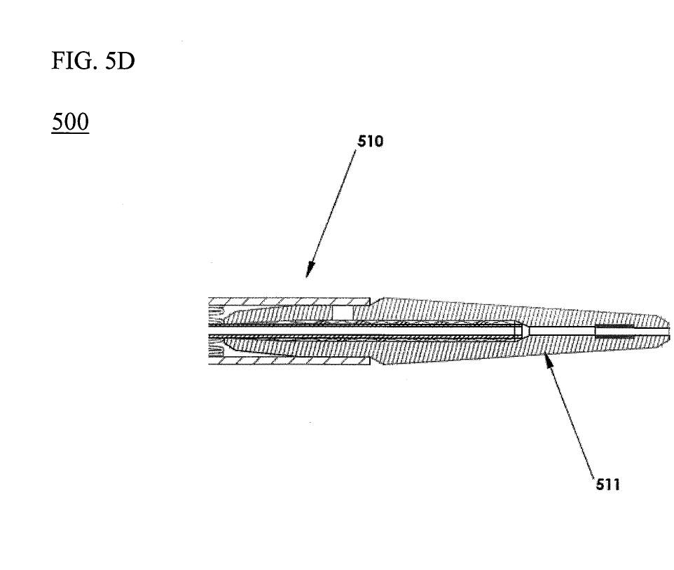

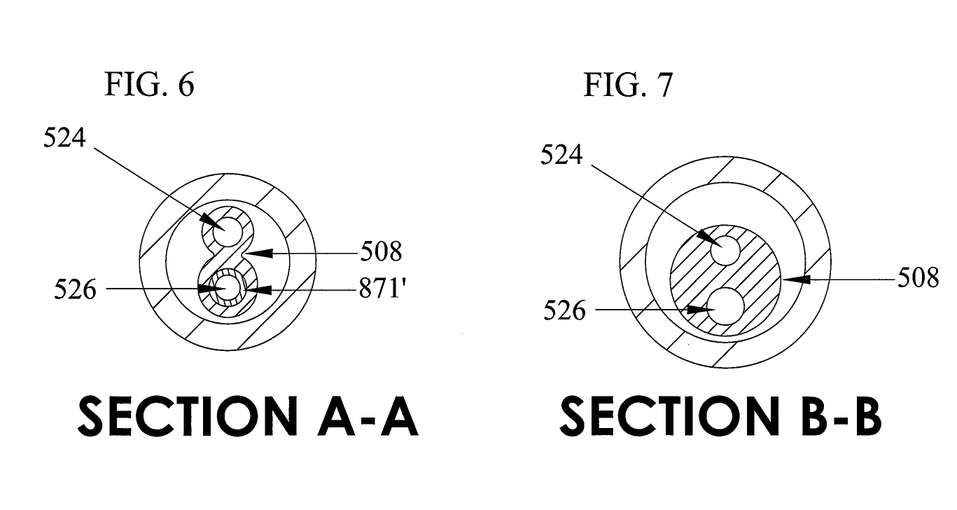

FIG. 5 schematically illustrates a portion of another embodiment of a catheter system 500. Catheter system 500 includes catheter body 510 (FIG. 5D) that defines a catheter inner lumen and a surrounding environment. Additionally, system 500 includes a dual lumen inner member 508 (FIG. 6, FIG. 7) disposed within the catheter inner lumen.

FIGS. 5 and 5B-5D illustrate the distal portion of a catheter, including the distal tip 511, the distal outer sheath 512, outer guide wire exit port 513 and an inner guide wire exit port 514. Specifically, FIG. 5 illustrates a cross sectional side view of a catheter 500, with FIGS. 5A-5D illustrating close up views of catheter 500, such that FIG. 5A illustrates the proximal portion of catheter 500, FIG. 5B illustrates the portion of catheter 500 immediately distal to FIG. 5A, FIG. 5D illustrates the distal portion of catheter 500, and FIG. 5C illustrates the portion of catheter 500 between FIGS. 5B and 5D. The structures illustrated in FIGS. 5 and 5A-5D are not to scale. Two flush ports 150′ and 150″ are illustrated in FIG. 5C although either fewer or more flush ports can be included. Additionally, FIG. 5C illustrates a stent 599 disposed in the catheter between the distal outer sheath 512 and inner member body 508.

Dual lumen inner member 508 includes a first lumen 524 and a second lumen 526. The first lumen 524 is parallel with the second lumen 526. The first lumen 524 is in fluidic communication with a fluid receptacle at the distal tip 511, so that when a flushing fluid is introduced into first lumen 524 at the distal tip of the catheter, the fluid displaces air from the guide wire lumen. Second lumen 526 is in fluid communication with a flush port in the handle and includes a hypotube 871′ (FIG. 6) for at least a portion of the length, and is in communication with and each flush port 150. Introduction of fluid at the handle then flushes the rest of the catheter. In the embodiment illustrated in FIG. 5, after a finger in placed over the (rapid exchange) outer guide wire exit port 513 the flushing fluid can be introduced at either or both the proximal and distal ends.

FIG. 6 illustrates a cross sectional view of catheter 500 taken at A-A, and FIG. 7 illustrates a cross sectional view of catheter 500 taken at B-B in FIG. 5C. FIG. 6 and FIG. 7 each show dual lumen inner member 508. In FIG. 6, hypotube 871′ is seen lining the second lumen 526.

The catheter 500 can be flushed in a one step flushing process, whereby a finger is placed over the outer guide wire exit port 513 flushing fluid is introduced either at the distal tip or at the proximal end. Catheter 500 is flushed in one step from either end of the catheter (from the tip or from the luer port at the handle) with a finger covering the RX wire exchange port. The flushing fluid flows from the tip through the guidewire lumen and around the stent and into the end of the hypotube as well as into the two flush ports 150′ and 150″ or alternately the flushing fluid flows from the luer fitting on the handle through the hypotube and out the end of the hypotube and the two flush ports 150′ and 150″ to bathe the stent and reach the guidewire lumen to pass through the guidewire lumen and out the end of the tip. Depending on the physical characteristics of the catheter (such as inner diameter and length), flushing performance may be improved by flushing from both the tip and luer ports. In such instances, each end can be flushed either simultaneously, or sequentially.

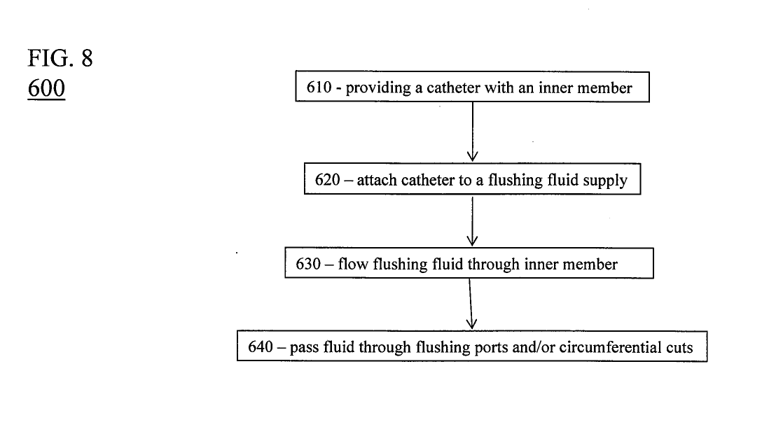

FIG. 8 illustrates the steps of a method 600 of flushing a catheter. Method 600 includes providing a catheter with an inner member during step 610. The inner member is implemented as inner members 100, 400, or 500. The catheter is attached, at step 620, to a flushing fluid supply, and the flushing fluid thereby flows under pressure, step 630, into the lumen of the inner member. In one example, the fluid is supplied from a syringe placed into fluid communication with a distal tip of the catheter. As it flows, the flushing fluid passes through at least one of the flushing ports and/or circumferential cuts in step 640. This passing of the flushing fluid flushes the catheter and inner member, as well as any stent or other medical device in the path of the fluid flow.

While specific embodiments according to the invention are disclosed herein, various changes and modifications can be made without departing from the spirit and scope of the invention.

1. A catheter inner member comprising:
an inner member body, including a first side and a second side, the second side opposing the first side, the inner member body including an inner surface defining an inner lumen, the inner member body further including an outer surface defining a surrounding environment;
at least a first flush port and a second flush port in the inner member body, the first flush port and second flush port providing fluid communication between the inner lumen and the surrounding environment, the first flush port disposed on the first side and the second flush port disposed on the second side, and wherein the first flush port is axially offset from the second flush port.

2. The catheter inner member of claim 1 further comprising a rapid exchange port having a block disposed within the inner member body and providing a fluid seal to reduce flow of the flushing fluid proximal of the rapid exchange port.

3. The catheter inner member of claim 1 further comprising an outer cover surrounding the inner member body.

4. The catheter inner member of claim 3 further comprising a self expanding stent contained by the outer cover and contained between the outer cover and the inner member body.

5. A catheter inner member comprising:
an inner member body, the inner member body defining an inner lumen and a surrounding environment;
at least a first incomplete circumferential cut providing fluid communication between the inner lumen and the surrounding environment.

6. The catheter inner member of claim 5 wherein the incomplete circumferential cut extends radially about the inner member body.

7. The catheter inner member of claim 5 wherein the incomplete circumferential cut extends spirally about the inner member body.

8. The catheter inner member of claim 5 further comprising:
a plurality of flush ports in the inner member body, the flush ports providing fluid communication between the inner lumen and the surrounding environment, the plurality of flush ports alternately disposed on a first side of the inner member body and a second side of the inner member body, and wherein the plurality of flush ports are axially displaced from each other.

9. The catheter inner member of claim 8 wherein the inner member body includes a flush port portion and a cut portion, wherein the cut portion is axially offset from the flush port portion, and wherein each flush port is disposed within the flush port portion, and wherein the at least a first incomplete circumferential cut is disposed within the cut portion.

10. The catheter inner member of claim 9 wherein the inner lumen is in fluid communication with a fluid source, and wherein the cut portion is disposed between the fluid source and the flush port portion.

11. A catheter system comprising:
a catheter including a catheter body defining a catheter inner lumen and a surrounding environment;
an inner member maintained within the catheter inner lumen, the inner member comprising:
an inner member body, including a first side and a second side, the second side opposing the first side, the inner member body defining an inner member inner lumen, the inner member body further including an outer surface; and
at least a first flush port and a second flush port in the inner member body, the first flush port and second flush port providing fluid communication between the inner member inner lumen and the catheter inner lumen, the first flush port disposed on the first side and the second flush port disposed on the second side, and wherein the first flush port is axially displaced from the second flush port.

12. The system of claim 11 further comprising:
a stent loaded in the catheter, the stent loaded adjacent at least one of the first flush port and second flush port.

13. The system of claim 12 further comprising:
at least a first incomplete circumferential cut providing fluid communication between the inner member inner lumen and the surrounding environment

14. The system of claim 13 wherein the inner member body includes a flush port portion and a cut portion, wherein the cut portion is axially offset from the flush port portion, and wherein each flush port is disposed within the flush port portion, and wherein the at least a first incomplete circumferential cut is disposed within the cut portion.

15. The system of claim 14 wherein the inner member inner lumen is in fluid communication with a fluid source, and wherein the cut portion is disposed between the fluid source and the flush port portion.

16. The system of claim 14 wherein the inner member inner lumen is in fluid communication with a fluid source, and wherein the flush port portion is disposed between the fluid source and the cut portion.

17. The system of claim 11 further comprising:
at least a first incomplete circumferential cut providing fluid communication between the inner member inner lumen and the surrounding environment

18. The system of claim 17 wherein the inner member body includes a flush port portion and a cut portion, wherein the cut portion is axially offset from the flush port portion, and wherein each flush port is disposed within the flush port portion, and wherein the at least a first incomplete circumferential cut is disposed within the cut portion.

19. The system of claim 18 wherein the inner member inner lumen is in fluid communication with a fluid source, and wherein the flush port portion is disposed between the fluid source and the cut portion

20. The system of claim 19 wherein the cut portion is disposed between the fluid source and the flush port portion.

21. A catheter inner member comprising:
an inner member body, the inner member body including an inner surface and an outer surface, the inner surface separated by the outer surface by a thickness;
at least a first incomplete circumferential cut, the at least a first incomplete circumferential cut extending from the outer surface radially a fraction of the thickness.

22. A catheter system comprising:
a catheter defining a lumen; and
a dual lumen inner member within the catheter, the dual lumen inner member defining a first lumen and a second lumen, the first lumen parallel with the second lumen, and wherein the first lumen is in fluid communication with a distal tip of the catheter, and wherein the second lumen is in fluid communication with a proximal end of the catheter, and wherein the second lumen includes at least a first flush port and a second flush port in the dual lumen inner member, the first flush port and second flush port providing fluid communication between the first lumen and a surrounding environment, the first flush port disposed on a first side and the second flush port disposed on a second side, and wherein the first flush port is axially offset from the second flush port.
